(19) United States
(12) Patent Application Publication
(10) Pub. No.: US 2021/0093521 A1
(43) Pub. Date: Apr. 1, 2021

(54) ANTIBACTERIAL ACTIVITY BY MG COMPLEX IN DEODORANT EMULSION FOR AEROSOLS, DEODORANT INCLUDING THE SAME AND USE OF THE DEODORANT

(71) Applicant: Henkel AG & Co. KGaA, Duesseldorf (DE)

(72) Inventors: INGA KERSTIN VOCKENROTH, Duesseldorf (DE); DANIEL SOLICH, Langenfeld (DE); IRIS MARINA STADLER, Duesseldorf (DE)

(73) Assignee: Henkel AG & Co. KGaA, Duesseldorf (DE)

(21) Appl. No.: 17/031,546

(22) Filed: Sep. 24, 2020

(30) Foreign Application Priority Data
Sep. 30, 2019 (DE) 10 2019 126 253.4

(51) Int. Cl. A61K 8/06; A61K 8/04; A61K 8/23; A61K 8/41; A61K 8/37; A61K 8/92; A61K 8/891

(52) U.S. Cl. CPC A61K 8/064; A61K 8/046; A61K 8/23; A61K 8/416; A61K 8/37; A61K 2800/805; A61K 8/891; A61K 2800/10; A61K 2800/30; A61K 2800/77; A61K 8/92

(57) ABSTRACT

The present disclosure concerns deodorant emulsion for an aerosol spray including magnesium sulfate and benzalkonium chloride, and a deodorant aerosol spray product comprising a pressure or pump spray container with a spray head, the filling of which includes the deodorant emulsion and optionally a propellant and/or further solvent, and a use of the deodorant aerosol spray product for deodorizing human skin.

### CROSS-REFERENCE TO RELATED APPLICATION

This application claims priority to German Patent Application No. 10 2019 126 253.4, filed Sep. 30, 2019, which is incorporated herein by reference in its entirety.

### TECHNICAL FIELD

The present disclosure concerns a deodorant emulsion for topical treatment of the skin, including magnesium sulphate and benzalkonium chloride.

### BACKGROUND

Washing, cleaning, and caring for your own body is a basic human need. The manufacturers of body hygiene products are constantly trying to meet these human needs in a variety of ways. Particularly important for daily hygiene is the continuous elimination or preventive prevention of the development of unpleasant body odor. Numerous special deodorizing body care products as well as body care products with antiperspirant effect are known in the state of the art, which were developed for the application in body care emitter with a high density of sweat glands, especially the armpit region or for example also the feet or palms of the hands.

Body odor is largely caused by the bacterial decomposition of individual components of sweat on the skin. When deodorizing the body, a rough distinction can be made between active substances that absorb substances that have already developed an unpleasant odor, e.g. activated carbon, zinc cicinoleate, cyclodextrins and ion exchangers, or cover them up, e.g. fragrances and perfumes, and active substances that prevent or at least slow down the decomposition of sweat and the development of unpleasant smelling substances, e.g. germ-inhibiting substances, prebiotically active components and enzyme inhibitors. Depending on the selected mechanism by which body odor is to be prevented, odor absorbers, fragrances, deodorizing ion exchangers, germ-inhibiting agents, prebiotically active components, enzyme inhibitors and other active substances can be used as cosmetic deodorizing agents. The active substances must be contained in the compositions, while ensuring that the compositions can be conveniently supplied in a desired pharmaceutical form and that the compositions do not cause an unpleasant feeling on the skin.

Many state-of-the-art cosmetic deodorants and/or antiperspirants used to inhibit perspiration and/or odor often contain aluminum and/or zirconium compounds as antiperspirant agents. On the one hand, these antiperspirant compounds reduce the body's sweat secretion by temporarily narrowing and/or clogging the ducts of the sweat glands, so that the amount of sweat can be reduced by from about 20 to about 60 percent. On the other hand, they have an additional deodorizing effect due to their antimicrobial effect.

However, the aluminum and/or zirconium compounds used to inhibit perspiration can lead to unpleasant skin reactions for some users. Aluminum compounds are often viewed critically by consumers. It has therefore become largely undesirable to use aluminum in cosmetic products, as there is a warning against excessive aluminum absorption into the human body due to the possibility of a health hazard. Aluminum, especially from aluminum salts, can be absorbed through the skin, sometimes with considerably better bioavailability than aluminum inevitably contained in food. For this reason, even some rather conservative officials or organizations dedicated to public health advise against the use of aluminum-containing cosmetics. In addition, the use of the antiperspirant compounds can lead to staining of clothing.

Furthermore, many users consider ethanol-free cosmetics to be desirable as ethanol is associated with skin dehydration and other skin irritation reactions. The technical field of this present disclosure is often referred to as non-alcoholic products, compositions, etc., although strictly speaking only ethanol-free products are meant, which may contain other alcohols. This terminology is not deviated from here. Thus, when a product or composition is described as alcohol-free, free of alcohols or similar, this should mean that no ethanol is contained, but other alcohols may be contained, unless explicitly stated otherwise at the appropriate place.

Sprays are a market-dominating category in the deodorant sector. Such products are therefore preferred by many users over sticks, roll-on deodorants, and other packaging. However, sprays pose challenges about the formulation of a deodorant, among other things because the propellants usually used are not compatible with many formulations, especially emulsions.

From the foregoing it follows that, in addition to the possibility of pressing the sweat formation to the lowest possible level, there is also the possibility of focusing not, or not exclusively, on reducing the sweat formation but, if necessary additionally, on preventing or reducing the formation of bad odors. Since normal, i.e. not excessive, sweating is often not even perceived as annoying as long as it is not accompanied by bad smell and some of the most effective antiperspirant active ingredients have fallen into disrepute, it follows that effective bad-odor limiting deodorants are desirable and correspond to the spirit of the times.

It should be noted at this point that, strictly speaking, the aim is not to prevent the formation and/or spread of malodorous substances, but that it is an alternative and/or additional way of preventing the perception of malodor, for example by adding to a deodorant ingredients which are capable of reducing or preventing the perception of malodor through superposition or, for example, desensitizing interaction with odor receptors. Thus, in the context of this present disclosure, bad odor reduction may also mean that the release of substances which smell bad under other circumstances is not prevented, but that it is merely prevented that these substances cause a bad odor impression.

Most of the experience gathered previously, for example, comes from oil sprays. It has been shown that the inhibition of the growth of certain bacterial species is an essential effective way to reduce unpleasant odor. However, it has been shown that conventionally proven antibacterial ingredients in the deodorant emulsion as contemplated herein are significantly less effective than in more traditional deodorant formulations or even show no effect at all against bacteria.

### BRIEF SUMMARY

The task underlying the present disclosure is thus to provide a deodorant emulsion which offers particularly effective inhibition of bacterial growth and which is suitable for an aerosol spray, and a corresponding spray product and its use. In an exemplary embodiment, provided is a deo-emulsion for an aerosol spray including magnesium sulphate and benzalkonium chloride. In particular, provided is a deo-emulsion for an aerosol spray including the magnesium sulfate in a concentration of from about 0.1 wt. % to about 10.0 wt, the benzalkonium chloride in a concentration of from about 0.05 wt. % to about 0.5 wt. %, and further including menthyl acetate in a concentration of from about 0.01% to about 0.5% by weight.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The task underlying the present disclosure is solved by the subjects described in detail below.

A deodorant emulsion for an aerosol spray is provided, comprising magnesium sulphate and benzalkonium chloride.

In experiments described in more detail in the further content of this patent application it was found that a combination of magnesium sulphate and benzalkonium chloride in the deodorant emulsions as contemplated herein inhibits to a particular extent the growth of bacteria which are associated with the development of bad body odor.

A deodorant emulsion within the meaning of this patent application is an emulsion containing only cosmetically acceptable ingredients. Otherwise, it is, according to the common definition of an emulsion, a finely divided mixture of two normally immiscible liquids without visible segregation, in which a finely divided mixture of two liquids, such as oil and water, is present. A liquid, in this context also called phase, forms small droplets, distributed in the other liquid. The phase that forms droplets is called internal phase or disperse phase. The phase in which the droplets float is called outer phase or continuous phase. Emulsions belong to the disperse systems and differ from mixtures of mixable liquids, such as ethanol and water. Emulsions are usually cloudy, milky liquids. According to present disclosure suitable deodorant emulsions are in the form of a water-in-silicone emulsion.

Until now, oil sprays were the usual products in the deodorant category of aerosol sprays, which dominates the market. However, it may be desirable to use an emulsion for a deodorant instead of a mixture with predominantly oily properties. This is desirable, for example, because certain ingredients cannot be worked into an oil spray but into an emulsion. However, the use of emulsions is made more difficult by the fact that they tend to be unstable and tend to separate back into their original phases. With the formulation described here, however, the inventors have found a way to produce an emulsion that is stable even in the presence of propellant gases and is therefore suitable for use in an aerosol spray. This stability is not only given in the presence of propellants, but also in the presence of common deodorants. The emulsion is also well-tolerated and can, for example, be applied directly after shaving without causing skin reactions and creates a pleasant feeling on the skin, especially no feeling of stickiness or oiliness. Furthermore, the emulsion is easy to spray. An emulsion also offers advantages in terms of resource consumption, as it typically includes a significant proportion of water instead of heavy and energy-intensive oily components. This is not only resource-saving in the provision of raw materials but also in wastewater treatment, which can be more effective with less pollution and with less energy input. Therefore, the deodorant emulsion as contemplated herein is advantageous under several aspects in terms of environmental protection.

In an embodiment the deodorant emulsion for an aerosol spray includes: the magnesium sulphate in a concentration from about 0.1% to about 10.0%, suitably from about 0.5% to about 7.0%, more suitably from about 1.0% to about 4.0%, based on the total weight of the deodorant emulsion for an aerosol spray, the benzalkonium chloride in a concentration from about 0.05% to about 0.5%, suitably from about 0.08% to about 0.35 wt. %, particularly suitably from about 0.1 wt. % to about 0.25 wt. %, based on the total weight of the deodorant emulsion for an aerosol spray, and suitably menthyl acetate in a concentration of from about 0.01 wt. % to about 0.5 wt. %, further suitably from about 0.03 wt. % to about 0.35-wt. %, particularly suitably from about 0.05 wt. % to about 0.2 wt. %, based on the total weight of the deodorant emulsion for an aerosol spray.

In these concentration ranges, the above-mentioned beneficial effects are particularly pronounced, while no excessive or wasteful amounts of the ingredients are consumed and there is considerable scope for shaping other parameters of the deodorant emulsion. If menthyl acetate is present, it can further reduce the level of unpleasant odor perception, especially in the defined concentration ranges.

In an embodiment the deodorant emulsion for an aerosol spray is free from aluminum salts, ethanol and/or decamethylcyclopentasiloxane and further includes at least one emulsifier, at least one salt, water, at least one silicone oil and at least one other oil.

It can be advantageous, as already mentioned at the beginning, that the deodorant emulsion for an aerosol spray is free of aluminum salts and ethanol. Ethanol can dry out the skin or cause other skin irritations and aluminum in deodorant products is sometimes not desired by users as human intake of aluminum should not exceed a certain level and, especially freshly shaved skin, allows aluminum to be absorbed from an applied cosmetic product. Apart from that, the defined deodorant emulsion allows a combination with common propellants without losing its stability, which is unusual and difficult to achieve, and it leaves a lot of scope for the further shaping of a deodorant based on it by further ingredients.

The present disclosure concerns embodiments based on a carrier including water or a water/alcohol mixture. Suitably the aqueous or aqueous-alcoholic carrier water or a water/alcohol mixture, the alcohol suitably being selected from a linear or branched alcohol such as, propanol, isopropanol, propanediol-1.3, dipropylene glycol, propanediol-12, propanediol-1.2. Suitably, water or the water/alcohol mixture is contained in an amount from about 60%-to about 99-%, by weight, more suitably from about 65%-to about 96-% by weight, most suitably from about 70%-to about 90-% by weight, based on the total weight of the deodorant emulsion.

In an embodiment the deodorant emulsion for an aerosol spray includes from about 0.5%-to about 5.0%-by weight, further suitably from about 1.0%-to about 4.5%-by weight, more suitably from about 2.0%-to about 4.0%-by weight, of at least one emulsifier, based on the total weight of the deodorant emulsion for an aerosol spray.

The exemplary properties described above are particularly pronounced in these concentration ranges and there is a particularly wide scope for shaping other parameters of the deodorant emulsion. Cosmetically acceptable emulsifiers are for example alkoxylated fatty acid alcohols like C14 to C18 alcohols with two to 22 ethoxy groups, for example steareth-2 or steareth-21, or PPG-15 stearyl ether. In the context of this present disclosure, a product commercially available as “Dow Corning ES-5227 DM Formulation Aid” is exemplary. It is a silicone emulsifier to produce low to medium viscosity water-in-silicone emulsions for a variety of applications such as color cosmetics, skin care, sun care, antiperspirants, and deodorants. It is an ethoxylated and propoxylated silicone emulsifier. Its main function is the production of water-in-silicone emulsions with textures from lotion to cream. It is a about 25% dispersion of silicone polyether in a low viscosity, non-volatile dimethicone liquid. INCI designation: Dimethicone (and) PEG/PPG-18/18 Dimethicone.

In an embodiment the deodorant emulsion for an aerosol spray includes from about 0.1%-to about 10.0%-by weight, further suitably from about 0.5%-to about 7.0%-by weight, more suitably from about 1.0%-to about 5.0%-by weight of at least one salt, based on the total weight of the deodorant emulsion for an aerosol spray.

Salts can fulfill various functions in deodorant emulsions. They can have an emulsion-stabilizing effect, can be antiperspirant or antibacterial, but are also used as moisturizers or in other functions. Their beneficial effects are particularly pronounced in the concentration ranges defined here. In principle, cosmetically acceptable salts may be used as long as they are not explicitly excluded here, for example silver salts such as silver citrate, dihydrogen silver citrate, silver lactate and silver sulphate, soluble complex salts of silver, colloidal silver and silver zeolites.

In an embodiment the deodorant emulsion for an aerosol spray includes from about 1.0%-to about 20.0%-by weight, further suitably from about 3.0%-to about 17.0%-by weight, more suitably from about 5.0%-to about 15.0%-by weight, of at least one silicone oil, based on the total weight of the deodorant emulsion for an aerosol spray.

The silicone oil can be selected from linear silicone oils, cyclic silicone oils, especially cyclopentasiloxane. Silicone oils give a pleasant silky skin feeling. Linear polydimethylsiloxanes bear the INCI designation Dimethicone. The chain length of the dimethylsiloxane chain determines the viscosity of the polydimethylsiloxanes. For the characterization of polydimethylsiloxanes or dimethicones their kinematic viscosity is therefore often given, usually in the unit centiStokes (cSt).

In an embodiment the deodorant emulsion for an aerosol spray includes from about 1.0%-to about 20.0%-by weight, further suitably from about 6.0%-to about 17.0%-by weight, more suitably from about 11.0%-to about 15.0%-by weight at least one other oil, based on the total weight of the deodorant emulsion for an aerosol spray.

The oil is involved in the formation of the oil phase. For example, the oil may be selected from paraffins, isoparaffins, polypropylene glycol ethers, polyethylene glycol ethers, organic esters, ethers, and ether carbonates. But basically, any cosmetically acceptable oil can be used.

On the one hand, the oil has the function of allowing certain ingredients to be incorporated into the deodorant emulsion, on the other hand it cares for the skin.

The oil phase may suitably contain particulate substances. Particularly suitable are fumed silica or layered silicates, which are modified to be even more hydrophobic. The particulate substances serve as thickeners and stabilizers. Hydrophobically modified bentonites and/or hydrophobically modified hectorites are exemplary.

In an embodiment the deodorant emulsion for an aerosol spray includes at least one perfume oil, further suitably in a concentration of from about 1.0%-to about 10.0%-by weight, further suitably from about 2.0%-to about 8.0%-by weight, more suitably from about 3.0%-to about 7.0%-by weight of the at least one perfume oil, based on the total weight of the deodorant emulsion for an aerosol spray.

A perfume oil includes one or more scents or fragrances. The definition of a fragrance for the purposes of the present application corresponds to the usual professional definition as given by ROMPP Chemie Lexikon (Römpp Lexikon Chemie', J. Falbe, M. Regitz (eds.), 10th edition, Thieme-Verlag). Examples of fragrance and aromatic compounds of the ester type are benzyl acetate, phenoxyethyl isobutyrate, p-tert. butylcyclohexyl acetate, linalyl acetate, dimethylbenzylcarbinyl acetate (DMBCA), phenylethyl acetate, benzyl acetate, ethylmethylphenylglycinate, allylcyclohexyl propionate, styrenallylpropionate, benzyl salicylate, cyclohexyl salicylate, floramate, melusate and jasmecyclate. Examples of fragrance and aromatic compounds of the ether type are benzyl ethyl ether and ambroxane, examples of fragrance and aromatic compounds of the aldehyde type are the linear alkanals with 8-18 C atoms, citral, citronellal, citronellyloxy-acetaldehyde, cyclamenaldehyde, lily and bourgeonal, examples of odoriferous compounds of the ketone type are jonones, alpha-isomethylionone and methylcedryl ketone, examples of odoriferous compounds of the alcohol type are anethole, citronellol, eugenol, geraniol, linalool, phenylethyl alcohol and terpineol, examples of odoriferous compounds of the terpene type are limonene and pinene. Examples of fragrance and scent compounds are pine, citrus, jasmine, patchouli, rose, ylang ylang oil, muscatel sage oil, chamomile oil, clove oil, mint oil, cinnamon leaf oil, lime blossom oil, juniper berry oil, vetiver oil, olibanum oil, galbanum oil, labdanum oil, orange blossom oil, neroli oil, orange peel oil and sandalwood oil, furthermore the essential oils like angelica root oil, anise oil, arnica blossom oil, basil oil, bay oil, bergamot oil, champaca blossom oil, silver fir oil, silver fir cone oil, elemi oil, eucalyptus oil, fennel oil, spruce needle oil, geranium oil, ginger grass oil, guaiac wood oil, gurjun balsam oil, helichrysum oil, ho-oil, ginger oil, Iris oil, cajeput oil, calamus oil, chamomile oil, camphor oil, canaga oil, cardamom oil, cassia oil, pine needle oil, copaiva balsam oil, coriander oil, spearmint oil, caraway oil, cumin oil, lavender oil, lemongrass oil, lime oil, tangerine oil, lemon balm oil, musk seed oil, myrrh oil, clove oil, niaouli oil, orange oil, origanum oil, Palmarosa oil, patchouli oil, Peru balsam oil, petitgrain oil, pepper oil, peppermint oil, pimento oil, pine oil, rose oil, rosemary oil, sandalwood oil, celery oil, spik oil, star anise oil, turpentine oil, thuja oil, thyme oil, verbena oil, juniper berry oil, wormwood oil, wintergreen oil, hyssop oil, cinnamon oil, citronella oil, lemon oil and cypress oil. Other fragrance and aroma compounds are ambrettolide, alpha-amyl cinnamaldehyde, anethole, anisaldehyde, anise alcohol, anisole, methyl anthranilic acid ester, acetophenone, benzyl acetone, Benzaldehyde, ethyl benzoate, benzophenone, benzyl alcohol, benzyl acetate, benzyl benzoate, benzyl formate, benzyl valerianate, borneol, bornyl acetate, alpha-bromostyrene, n-decylaldehyde, n-dodecyl aldehyde, eugenol, eugenol methyl ether, eucalyptol, farnesol, fenchone, fenchyl acetate, geranyl acetate, geranyl formate, heliotropin, methyl heptanecarboxylate, heptaldehyde, Hydroquinone dimethyl ether, hydroxycinnamic aldehyde, hydroxycinnamic alcohol, indole, iron, isoeugenol, isoeugenol methyl ether, isosafrol, jasmone, camphor, carvacrol, carvone, p-cresol methyl ether, coumarin, p-methoxyacetophenone, methyl-n-amylketone, methyl anthranilic acid methyl ester, p-methylacetophenone, methylchavicol, p-methylquinoline, methyl-β-naphthylketone, methyl-n-nonyl acetaldehyde, Methyl-n-nonylketone, muscone, β-naphthol ethyl ether, β-naphthol methyl ether, nerol, nitrobenzene, n-nonylaldehyde, nonylacohol, n-octylaldehyde, p-oxy-acetophenone, pentadecanolide, β-phenylethyl alcohol, phenylacetaldehyde dimethyacetal, phenylacetic acid, pulegone, safrole, isoamyl salicylic acid ester, methyl salicylic acid ester, hexyl salicylic acid ester, cyclohexyl salicylic acid ester Santalol, skatole, terpineol, thyme, thymol, y-undecalactone, vanillin, veratrum aldehyde, cinnamic aldehyde, cinnamic alcohol, cinnamic acid, ethyl cinnamate and benzyl cinnamate. Other (more volatile) fragrances are alkyl isothiocyanates (alkyl legumes), butanedione, limonene, linalool, linayl acetate and propionate, menthol, menthone, methyl-n-heptenone, phellandrene, phenylacetaldehyde, terpinyl acetate, citral and citronellal. An exemplary perfume oil in the context of the present disclosure is the commercially available product “Parfum Crispy Grapefruit A T12033987”.

A perfumed deodorant has the advantage that it not only reduces or prevents the emergence and/or spread of bad smelling substances, but also gives the wearer a subtle pleasant scent without the need to use another product.

In an embodiment the deodorant emulsion for an aerosol spray includes at least one deodorant active ingredient, further suitably in a concentration of from about 0.1%-to about 50.0%-by weight, further suitably from about 0.5%-to about 40.0%-by weight, particularly suitably from about 1.0%-to about 30.0%-by weight of the at least one deodorant active ingredient, based on the total weight of the deodorant emulsion for an aerosol spray.

Antimicrobial, antibacterial or germ-inhibiting substances, antioxidants, odor adsorbents or enzyme inhibitors can be used as such additional deodorants or deodorant active ingredients. Suitable antimicrobial, antibacterial, or germ-inhibiting substances are organohalogen compounds as well as organohalides, quaternary ammonium compounds and several plant extracts. Suitable are halogenated phenol derivatives such as hexachlorophene orrgasan DP 300 (triclosan, 2.4.4′-trichloro-2′-hydroxydiphenyl ether), 3.4.4′-trichlorocarbanilide, chlorhexidine (1.1′-hexamethylene-bis-[5-(4-chlorophenyl)]-biguanide), chlorhexidine gluconate, benzalkonium halides and cetylpyridinium chloride. Furthermore, sodium bicarbonate and sodium phenolsulfonate as well as e.g. the components of lime blossom oil can be used. Weaker antimicrobial substances, which however have a specific effect against the gram-positive germs responsible for sweat decomposition, can also be used as deodorant agents. Benzyl alcohol can also be used as deodorant active ingredient. Other antibacterial deodorants are lantibiotics, glycoglycerolipids, sphingolipids (ceramides), sterols and other active ingredients that inhibit bacterial adhesion to the skin, e.g. glycosidases, lipases, proteases, carbohydrates, di- and oligosaccharide fatty acid esters and alkylated mono- and oligosaccharides. Suitable deodorant active ingredients are long-chain diols, e.g. 1,2-alkane-(C5-C18) diols, glycerol mono(C8-C18) fatty acid esters or, in an embodiment, glycerol mono(C6-C16) alkyl ethers, especially 2-ethylhexyl glycerol ethers, which are very well tolerated by the skin and mucous membranes and are effective against corynebacteria, and also phenoxyethanol, phenoxyisopropanol (3-phenoxy-propan-2-ol), anisyl alcohol, 2-methyl-5-phenyl-pentan-1-ol, 1.1 dimethyl-3-phenyl-propan-1-ol, benzyl alcohol, 2-phenylethane-1-ol, 3-phenylpropane-1-ol, 4-phenyl-butan-1-ol, 5-phenylpentane-1-ol, 2-Benzylheptan-1-ol, 2,2-dimethyl-3-phenyl-1-ol, 2.2-Dimethyl-3-(3′-methylphenyl) propan-1-ol, 2-ethyl-3-phenylpropan-1-ol, 2-ethyl-3-(3′-methylphenyl) propan-1-ol, 3-(3′-chlorophenyl)-2-ethylpropane-1-ol, 3-(2′-chlorophenyl)-2-ethylpropane-1-ol, 3-(4′-chlorophenyl)-2-ethylpropane-1-ol, 3-(3 ‘, 4’-dichlorophenyl)-2-ethylpropane-1-ol, 2-Ethyl 3-(2′-methylphenyl) propan-1-ol, 2-ethyl-3-(4′-methylphenyl) propan-1-ol, 3-(3 ‘, 4’-dimethylphenyl)-2-ethylpropane- 1-ol, 2-ethyl-3-(4′-methoxyphenyl) propan-1-ol, 3-(3 ‘, 4’-dimethoxyphenyl)-2-ethylpropane-1-ol, 2-allyl-3 phenylpropane-1-ol and 2-n-pentyl-3-phenyl-propan-1-ol.

As contemplated herein, the suitable deodorant active ingredients are odor absorbers, deodorizing ion exchangers, germ-inhibiting agents, prebiotically active components as well as enzyme inhibitors or, in an embodiment, combinations of the named active ingredients. Silicates serve as odor absorbers, which at the same time favorably support the rheological properties of the composition as contemplated herein. As contemplated herein, suitable silicates are phyllosilicates, montmorillonite, kaolinite, ilite, beidellite, nontronite, saponite, hectorite, bentonite, smectite, calcium silicates and talc.

Other suitable odor absorbers include zeolites, zinc cicinoleate, cyclodextrins, certain metal oxides and chlorophyll. Other odor absorbers suitable for use in the present disclosure are selected from pearlite. As contemplated herein, germ-inhibiting or antimicrobial agents are understood to be those agents which reduce the number of skin germs involved in the formation of odors or inhibit their growth. These germs include various species from the group of staphylococci, the group of corynebacteria, anaerocococci and micrococci.

As contemplated herein, organohalogen compounds as well as organohalides, quaternary ammonium compounds, several plant extracts and zinc compounds are suitable as germicidal or antimicrobial agents. These include triclosan, chlorhexidine and chlorhexidine gluconate, 3,4,4′-trichlorocarbanilide, bromochlorophene, dichlorophene, chlorothymol, chloroxylenol, hexachlorophene, dichloro-m-xylenol, dequalinium chloride, domiphene bromide, Ammonium phenolsulfonate, benzalkonium halides, benzalkonium cetyl phosphate, benzalkonium saccharinates, benzethonium chloride, cetylpyridinium chloride, laurylpyridinium chloride, laurylisoquinolinium bromide, methylbenzethonium chloride. Furthermore, phenol, phenoxyethanol, disodium dihydroxyethylsulfosuccinylundecylenate, sodium bicarbonate, zinc lactate, sodium phenolsulfonate and zinc phenolsulfonate, ketoglutaric acid, terpene alcohols such as farnesol, chlorophyllin-copper complexes, a monoalkyl glycerol ether with a branched or linear saturated or unsaturated, optionally hydroxylated C6-C22 alkyl radical, particularly suitably α-(2-ethylhexyl)glycerol ether, commercially available as Sensiva® SC 50 (ex Schülke & Mayr), carboxylic acid esters of mono-, di- and triglycerol (e.g. (e.g. glycerol monolaurate, diglycerol monocaprinate), lantibiotics and plant extracts (e.g. green tea and components of lime blossom oil).

Further suitable deodorant active substances are selected from so-called prebiotically active components, by which as contemplated herein such components are to be understood which only or at least predominantly inhibit the odor-forming germs of the skin microflora, but not the desired, i.e. the non-odor-forming germs which belong to a healthy skin microflora. Explicit mention must be made here of active substances such as conifer extracts, in particular from the group of Pinaceae, and plant extracts from the group of Sapindaceae, Araliaceae, Lamiaceae and Saxifragaceae, in particular extracts of Picea spp., Paullinia sp., Panax sp., Lamium album or Ribes nigrum, and mixtures of these substances.

Other suitable deodorant active ingredients are selected from the germ-inhibiting perfume oils and the Deosafe® perfume oils available from Symrise, formerly Haarmann and Reimer. Other suitable deodorant active substances are selected from silver salts, silver citrate, dihydrogen silver citrate, silver lactate and silver sulphate, soluble complex salts of silver, colloidal silver, and silver zeolites.

Enzyme inhibitors include substances which inhibit the enzymes responsible for sweat decomposition, arylsulfatase, β-glucuronidase, aminoacylase, esterases, lipases and/or lipoxigenase, e.g. trialkyl citric acid esters, triethyl citrate, or zinc glycinate.

Suitable deodorant emulsions as contemplated herein at least one deodorant active substance is selected from arylsulfatase inhibitors, beta-glucuronidase inhibitors, aminoacylase inhibitors, esterase inhibitors, Lipase inhibitors and lipoxigenase inhibitors, a monoalkylglycerol ethers with a branched or linear saturated or unsaturated, optionally hydroxylated C6-C22 alkyl radical, in particular α-(2-ethylhexyl) glycerol ether, phenoxyethanol, benzylheptanol, Cocamidopropyl PG-Dimonium Chloride Phosphates and Butyloctanoic acid, Polyglycerol-3-Caprylates, antibacterial perfume oils, Deosafe® perfume oils (Deosafe® is a registered trademark of Symrise, formerly Haarmann & Reimer), prebiotically active components, trialkyl citric acid esters, in particular triethyl citrate, active substances which reduce the number of skin germs from the group of staphylococci, corynebacteria, anaerocococci and micrococci involved in the formation of odors, or inhibit their growth, zinc compounds, in particular zinc phenolsulphonate and zinc cicinoleate, organohalogen compounds, in particular triclosan, chlorhexidine, chlorhexidine gluconate and benzalkonium halides, quaternary ammonium compounds, in particular cetylpyridinium chloride, odor absorbers, in particular silicates and zeolites, sodium bicarbonate, lantibiotics, and mixtures of the aforementioned substances.

The use of such active ingredients in the deodorant emulsion is advantageous, as they can give a deodorant additional effect as described in connection with the respective substances.

It is also exemplary that the deodorant emulsion for an aerosol spray is free of decamethylcyclopentasiloxane.

Many cosmetics, especially deodorants and antiperspirants, contain the chemical compound decamethylcyclopentasiloxane, in short siloxane D5, as solvent. This siloxane is a volatile silicon compound and many users are concerned about its possible health effects. There is also controversy as to whether the substance should be classified as a PBT, i.e. as a persistent, bio-accumulative, and toxic substance that accumulates in the environment and whose effect on humans, if any, would be difficult to assess.

An already known problem is the accumulation of siloxanes in sewage and landfill sludge. The compound enters these via the sewage and waste cycle. During the combustion of sewage and landfill gases, e.g. in gas engines for energy generation, silica (“sand”) is produced, which leads to premature wear of the combustion engines and to high costs for gas treatment.

For the reasons given above, it is desirable to dispense with decamethylcyclopentasiloxane and the inventors have succeeded in providing a formulation which makes this possible.

Furthermore, the present disclosure relates to a deodorant aerosol spray product including a pressure or pump spray container with a spray head, the filling of which includes a deodorant emulsion as contemplated herein as contemplated herein and optionally a propellant and/or further solvent.

The provision of the aerosol spray product as contemplated herein has the advantage that the advantages described above can be experienced by a user in a particularly practical way that is suitable for everyday use. For example, many users appreciate the fresh feeling when spraying and the even distribution.

Furthermore, the present disclosure relates to the use of a deodorant aerosol spray product for deodorizing human skin, as contemplated herein present disclosure.

Furthermore, the present disclosure relates to a non-therapeutic, cosmetic method for deodorizing human skin, in which a deodorant emulsion as contemplated herein is applied to human skin. Furthermore, the present disclosure relates to a non-therapeutic, cosmetic method for deodorizing human skin, in which a deodorant emulsion as contemplated herein present disclosure is sprayed onto human skin by employing a deodorant aerosol spray product as contemplated herein present disclosure.

The use as contemplated herein or the use as contemplated herein as well as the deodorization process as contemplated herein or the deodorization process as contemplated herein have the advantage that the advantages described above can be experienced by a user in a particularly practical and everyday way. For example, many users appreciate the fresh feeling when spraying and the even distribution.

Unless otherwise indicated in detail at the relevant place, the following definitions shall apply to the context of the present disclosure:

The term antiperspirant deodorant is not used quite uniformly in the field of cosmetics. Basically, an antiperspirant is usually understood to be a product designed to reduce or stop sweating and a deodorant is usually understood to be a product designed to reduce or stop the spread of unpleasant odors, for example by preventing the formation of bad smelling substances or by superimposing odors, binding or decomposing them.

The statement “free from” and comparable statements should mean that a substance, mixture of substances or product to which the statement relates is essentially free of the substance to which the statement also relates, i.e. to the extent that the thematic advantageous effect is achieved or as far as free of it, as is common in the technical field and can be achieved with the usual technical effort.

In the case of compounds of which both salt and acid forms are common, the specification of one form should include the other form. For example, the following shall apply: N,N-bis(carboxylatomethyl)-L-glutamate and/or methylglycinediacetic acid include the respective salt forms, in sodium salts and acid forms.

A spray product in the context of the present disclosure, an aerosol spray product, is a spray product which generates an aerosol. It can be a spray product with propellant, which is common in the deodorant sector, or a pump spray product, which is less common but preferred by certain users, for example because it is easier to transport, for example when travelling by air, or because it is generally preferred to release as little propellant as possible into the living environment and, ultimately, the atmosphere.

A salt as contemplated herein is any cosmetically acceptable salt. Compounds that are in salt form, but where properties dominate that are not due to their salt property, can be designated differently and placed in a different category without negating their salt property or changing the definition of a salt.

If at one point of this patent application an ingredient is mentioned which can be assigned to a certain substance class, e.g. salts, but which is dealt with on its own or in a smaller group, and at another point comments are made on just this substance class, this may mean, depending on the wording, that an additional representative of the substance class must be present or that the respective condition can also be fulfilled by the aforementioned substance alone.

As previously disclosed, the inventors have developed a new emulsion-based deodorant spray formulation to prevent body odor by reducing bacterial growth. A complex of magnesium sulphate and benzalkonium chloride was added to inhibit the growth of bacteria that cause body odor. The targeted growth inhibition was verified with different methods, as described in detail below.

The inhibition yard test (or agar diffusion test) is used to investigate the growth-inhibiting effect of substances or mixtures of substances on the most important bacterial germs known to cause body odor. For this purpose, the pathogen-containing material is spread out on a culture medium (usually on an agar layer within a Petri dish). Filter paper discs impregnated with the mixture of active substances to be tested or with a control without an active substance are placed on the bacterial layer so that the active substances can penetrate the material and spread there. If the test substance has an antimicrobial effect, clearly visible circles, so-called inhibition yards, form around the filter paper discs due to the inhibition of the pathogen growth. These can be measured in mm diameter.

An inhibition yard test was performed with deodorant emulsions containing the following potential or actual bacterial growth inhibiting additives (the amounts are given in weight %)

| | | | | | | | | Result | |
|---|---|---|---|---|---|---|---|---|---|
| | Phenoxy | Symdiol | Isocarb | Barquat | EDETA BX | Dissolvins | | Staph epid. | Staph hominis |
| Sample | ethanol | 68T | 12 | DM-50 EP | Powder | GL-47-S | MgSO4 | DSM 20042 | DSM 20328 |
| a | 1 | 0.5 | | | | | 2.5 | no | no |
| b | 1 | 0.5 | 0.35 | | | | 2.5 | no | no |
| c | 1 | 0.75 | | | | | 2.5 | no | no |
| d | 1 | | 0.35 | | | | 2.5 | no | no |
| e | 1 | 0.5 | 0.7 | | | | 2.5 | no | no |
| f | 1 | | | 0.19 | 0.2 | | 2.5 | 3.7 mm +− | 4.3 mm |
| | | | | | | | | 0.4 mm | |

Known antibacterial agents such as symdiol (1,2-hexanediol+octanediol+tropolone) and isocarb (2-butyloctanioc acid) obviously have no effect in the deodorant emulsion, although their effect is known and described in other formulations. They work neither individually nor in combination.

Benzalkonium chloride, present in the form of the commercially available product “Barquat DM-50 EP”, on the other hand, shows a good effect even in small quantities, as can be seen from the large inhibition zone for both germs tested. In this formulation it was tested with EDTA, but in a further test, described below, an additional effect of EDTA was refuted.

The antibacterial effectiveness of various deodorant formulations has been tested against four bacteria that cause body odor using a membrane test. The tests showed excellent effectiveness of the tested formulations containing Barquat. The formula containing only phenoxyethanol was not effective.

Sweat, as it is secreted from the armpits, is odorless. It is known that the typical strong underarm odor can only be released from apocrine secretions and that the action of skin bacteria and hydrolytic bacterial enzymes is required to release odorous compounds from non-odorous precursors present in these secretions. In fact, the axilla is a skin region exemplified by a dense bacterial population dominated by the genera Staphylococcus and Corynebacterium.

Most individuals carry flora dominated by one of these genera, and a strong correlation has been found between high cell densities of staphylococci or corynebacteria and strong body odor.

Consequently, cosmetic formulations designed to prevent the formation of body odor usually contain active ingredients that have an antimicrobial effect on bacteria that cause body odor.

Data on the effectiveness of several degassed AP aerosol formulations against four selected skin bacteria are presented here. The test bacteria were selected for their key role in the formation of body odors. The membrane test was used because it mimics to some extent the microbiological situation under the armpit.

In this test system the odor causing bacteria were species belonging to the bacterial genera of Staphylococcus, Corynebacterium and Anaerococcus. The test bacteria were selected for their key role in the formation of body odors, as described by Egert et al. 2014 (Markus Egert et al.: In Deo Veritas —, 2014, Biospektrum 05.14, S. 497-499)

Typically, the bacteria were placed on tissue culture inserts, i.e. on a membrane surface, and incubated for 3 hours. This imitates the underarm skin colonized by bacteria. Incubations were supplemented with 30 μl of the product and the membrane is surrounded by artificial Eccrin sweat. After about 14 h incubation bacteria were washed off the membranes and quantified by cell counting on agar plates. The results are presented as logarithmic reduction factors calculated in comparison to a control.

The membrane test simulates armpit-like surface conditions in terms of temperature, humidity, bacteria density, and amount of active ingredient per surface.

The test parameters for the TCI Leave-On Membrane model were:

a) Apply 30 μl product per membrane while shaking at 1000 rpm
b) incubate at room temperature for 15 minutes to evaporate the solvent
c) Application of 40 μl Test germ mixture per membrane while shaking at 1000 rpm
i. Initial cell count per membrane: ˜4.6×10⁶-2.0×10⁷ cfu (depending on the strain)
(d) incubation for 14 h at 37° C.
(e) harvesting of bacterial remains with 3 ml inactivator solution
(f) Cultivation and quantification of all bacteria on agar plates
(g) Control: water instead of formulation
(h) Results: log reduction factor compared to the start inoculum

When test germs were used:

(a) Staphylococcus epidermidis DSMZ 20042
(b) Staphylococcus hominis DSMZ 20238
(c) Anaerococcus octavius DSMZ 11663
(d) Corynebacterium tuberculostearicum isolate 121

The formulation was tested for long-term antimicrobial efficacy (14 h) against relevant axillary bacteria. A bacterial reduction of 2-3 log is usually accepted as a significant antimicrobial effect. The results are given in the following table (the quantities are given in weight %)

| | | | | | | | | Result | | | |
|---|---|---|---|---|---|---|---|---|---|---|---|
| | | | | | | | | | | C. | |
| | Phenoxy | Symdiol | Isocarb | Barquat | EDETA BX | Dissolvins | | S. | S. | tuberculo- | A. |
| | ethanol | 68T | 12 | DM-50 EP | Powder | GL-47-S | MgSO4 | epidermidis | hominis | stearicum | octavius |
| g | 1 | 0.5 | | | | | 2.5 | 0.72 | 0 | 1.02 | 0.72 |
| h | 1 | | | 0.18 | | 0.18 | 2.5 | 5.21 | 4.33 | 5.61 | 5.21 |
| i | 1 | | | 0.18 | 0.1 | | 2.5 | 7.00 | 5.71 | 7.54 | 7.00 |
| j | 1 | | | 0.18 | | | 2.5 | 7.00 | 6.09 | 7.54 | 7.00 |
| k | | | | | | | | 0.65 | 0 | 0.93 | 0.65 |

The formulations containing barquat were thus able to reduce the number of bacteria in the tested species by at least >5 log when tested for 14 h. The non-barquate-containing formulations could not significantly reduce the number of bacteria in the tested species.

So, it can be recorded: Symdiol shows no effect in the membrane test. Barquat shows a good effect in combination with EDDS and EDTA. However, it shows the same good effect even without the complexing agents.

An invented example of a deodorant emulsion is:

| | Percentage |
|---|---|
| Ingredient | [weight %] |
| Triethyl citrate | 7.42 |
| 2-Ethylhexyl palmitate | 6.00 |
| Dimethicone 5 cSt | 10.00 |
| Phenoxyethanol, pure | 1.00 |
| 950313 Symfresh Alpha | 0.10 |
| Dow Corning ES-5227 DM Formulation Aid | 2.80 |
| Water, demineralized | 30.00 |
| Magnesium sulfate 7H2O extra pure | 2.50 |
| Barquat DM-50 EP | 0.18 |
| Propanediol-1.2 | 35.00 |
| Perfume | 5.00 |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A deodorant-emulsion for an aerosol spray comprising magnesium sulphate and benzalkonium chloride.

2. The deodorant-emulsion according to claim 1, comprising:
the magnesium sulfate in a concentration of from about 0.1 wt. % to about 10.0 wt,
the benzalkonium chloride in a concentration of from about 0.05 wt. % to about 0.5 wt. %, and
further comprising menthyl acetate in a concentration of from about 0.01% to about 0.5% by weight, based on the total weight of the deodorant emulsion.

3. The deodorant-emulsion according to claim 1, wherein the deodorant emulsion is free of aluminum salts, ethanol and/or decamethylcyclopentasiloxane, and further comprises:
at least one emulsifier,
at least one salt,
water,
at least one silicone oil, and
at least one further oil.

4. The deodorant-emulsion according to claim 3, comprising from about 0.5%-to about 5.0%-by weight, of the at least one emulsifier, based on the total weight of the deodorant-emulsion.

5. The deodorant-emulsion according to claim 3, comprising from about 0.1%-to about 10.0%-by weight, of the at least one salt, based on the total weight of the deodorant-emulsion.

6. The deodorant-emulsion according to claim 3, comprising from about 1.0%-to about 20.0%-by weight, of the at least one silicone oil, based on the total weight of the deodorant emulsion.

7. The deodorant-emulsion according to claim 3, comprising from about 1.0%-to about 20.0%-by weight, of at least one further oil, based on the total weight of the deodorant emulsion.

8. The deodorant-emulsion according to claim 1, wherein the deodorant emulsion further comprises at least one perfume oil, based on the total weight of the deodorant emulsion of an aerosol spray and/or wherein the deodorant emulsion further comprises at least a deodorant active by weight of at least one deodorant active substance, based on the total weight of the deodorant emulsion.

9. The deodorant emulsion according to claim 1, wherein the deodorant-emulsion comprises water or a water/alcohol mixture.

10. The deodorant-emulsion according to claim 9, wherein the water or the water/alcohol mixture is included in an amount of from 60%-to 99%-by weight, based on the total weight of the deodorant-emulsion.

11. The deodorant-emulsion according to claim 1, wherein the deodorant-emulsion further comprises a silicone emulsifier for the preparation of water-in-silicone emulsions, which is selected from an about 25% dispersion of silicone polyether in a low-viscosity, non-volatile dimethicone liquid with the INCI name Dimethicone (and) PEG/PPG-18/18 Dimethicone.

12. The deodorant-emulsion according to claim 1, comprising:
the magnesium sulfate in a concentration of from about 0.5 wt. % to about 7.0 wt,
the benzalkonium chloride in a concentration of from about 0.08 wt. % to about 0.35 wt. %, and
the menthyl acetate in a concentration of from about 0.03% to about 0.35% by weight, based on the total weight of the deodorant emulsion for an aerosol spray.

13. The deodorant-emulsion according to claim 1, comprising:
the magnesium sulfate in a concentration of from about 1.0 wt. % to about 4.0 wt,
the benzalkonium chloride in a concentration of from about 0.1 wt. % to about 0.25 wt. %, and
the menthyl acetate in a concentration of from about 0.05% to about 0.2% by weight, based on the total weight of the deodorant emulsion for an aerosol spray.

14. The deodorant-emulsion according to claim 3, comprising from about 2.0%-to about 4.0%-by weight, of at the least one emulsifier, based on the total weight of the deodorant-emulsion.

15. The deodorant-emulsion according to claim 3, further comprising from about 0.5%-to about 7.0%-by weight, of the at least one salt, based on the total weight of the deodorant-emulsion.

16. The deodorant-emulsion according to claim 3, comprising from about 1.0%-to about 20.0%-by weight, of the at least one silicone oil, based on the total weight of the deodorant emulsion.

17. The deodorant-emulsion according to claim 3, comprising from about 6.0%-to about 17.0%-by weight, of at least one further oil, based on the total weight of the deodorant emulsion.

18. The deodorant-emulsion according to claim 9, wherein the water or the water/alcohol mixture is included in an amount of from 70%-to 90%-by weight, based on the total weight of the deodorant emulsion.

19. A deodorant aerosol spray product comprising a pressure or pump spray container with spray head, the filling of which comprises the deodorant-emulsion according to claim 1 and optionally a propellant and/or further solvent.

20. A non-therapeutic cosmetic method for deodorizing human skin comprising applying to the human skin the deodorant aerosol spray product according to claim 19.
